(19) United States
(12) Patent Application Publication
(10) Pub. No.: US 2018/0000696 A1
(43) Pub. Date: Jan. 4, 2018

(54) AROMATHERAPY ADHESIVE STRIP

(71) Applicants: Scott Villeneuve, Orlando, FL (US); Melanie Villeneuve, Orlando, FL (US)

(72) Inventors: Scott Villeneuve, Orlando, FL (US); Melanie Villeneuve, Orlando, FL (US)

(21) Appl. No.: 15/588,456

(22) Filed: May 5, 2017

Related U.S. Application Data
(60) Provisional application No. 62/332,009, filed on May 5, 2016.

(51) Int. Cl. A61K 8/02; A61K 9/70; A61K 9/00

(52) U.S. Cl. CPC A61K 8/0208; A61K 9/007; A61K 9/70

(57) ABSTRACT

An aromatic adhesive strip that includes: a first end; a second end; an absorbent center extending between the first end and second end; an adhesive surface on a rear side of the adhesive strip; and a release paper covering the adhesive surface, where the release paper is adapted to be removed to apply the adhesive strip to an upper lip of a user. The absorbent center includes a cotton pad, wherein the cotton pad includes a liquid aromatic oil and the liquid aromatic oil is adapted to diffuse a scent over a predetermined time period. The absorbent center is preferably surrounded by non-absorbent strip material, where the non-absorbent material is adapted to retain the absorbent center in place.

### CROSS REFERENCE TO OTHER APPLICATIONS

This application claims priority to U.S. Provisional Application Ser. No. 62/332,009 filed on May 5, 2016.

### BACKGROUND OF THE INVENTION

### Field of the Invention

This present invention relates generally to respiratory devices and, more particularly, to an aromatherapy adhesive strip having an intended placement under a wearer's nose.

### Description of the Prior Art

The use of various aromatic plant oils for therapeutic purposes, including for respiratory disinfection, decongestant, expectoration is well known. A problem which still exists, however, is that when the delivery such aromatics to a user's sinus area is not optimized, positive outcomes from such therapy may be reduced. Thus, there remains a need for an aromatherapy adhesive strip which could be positioned directly under a user's nose and avail an aromatic for direct inhalation. It would be helpful if such an aromatherapy adhesive strip included an absorbent center reservoir for securely holding a supply of an aromatic being diffused.

The Applicant's invention described herein provides for an aromatherapy adhesive strip adapted to hold an aromatic substance and be worn by a user directly under their nose. When in operation, the aromatherapy adhesive strip enables more effective and efficient delivery of aromatics to a user for not just therapy but also for the purpose of altering one's mood, cognitive, psychological or physical well-being. As a result, many of the limitations imposed by prior art structures are removed.

### SUMMARY OF THE INVENTION

The present invention relates to an aromatic adhesive strip that includes: a first end; a second end; an absorbent center extending between the first end and second end; an adhesive surface on a rear side of the adhesive strip; and a release paper covering the adhesive surface, where the release paper is adapted to be removed to apply the adhesive strip to an upper lip of a user.

The absorbent center includes a cotton pad, wherein the cotton pad includes a liquid aromatic oil and the liquid aromatic oil is adapted to diffuse a scent over a predetermined time period. The absorbent center is preferably surrounded by non-absorbent strip material, where the non-absorbent material is adapted to retain the absorbent center in place.

### BRIEF DESCRIPTION OF THE DRAWINGS

FIG. 1A is a front view of an aromatherapy adhesive strip in accordance with the present invention.

FIG. 1B is a back view of the aromatherapy adhesive strip in accordance with the present invention.

FIG. 1C is a sectional view of the aromatherapy adhesive strip in accordance with the present invention.

FIG. 2 is a front view of the aromatherapy adhesive strip in accordance with the present invention in place on a wearer.

FIG. 3 is a side view of an aromatherapy adhesive strip in accordance with the present invention in place on a wearer.

### DETAILED DESCRIPTION OF THE INVENTION

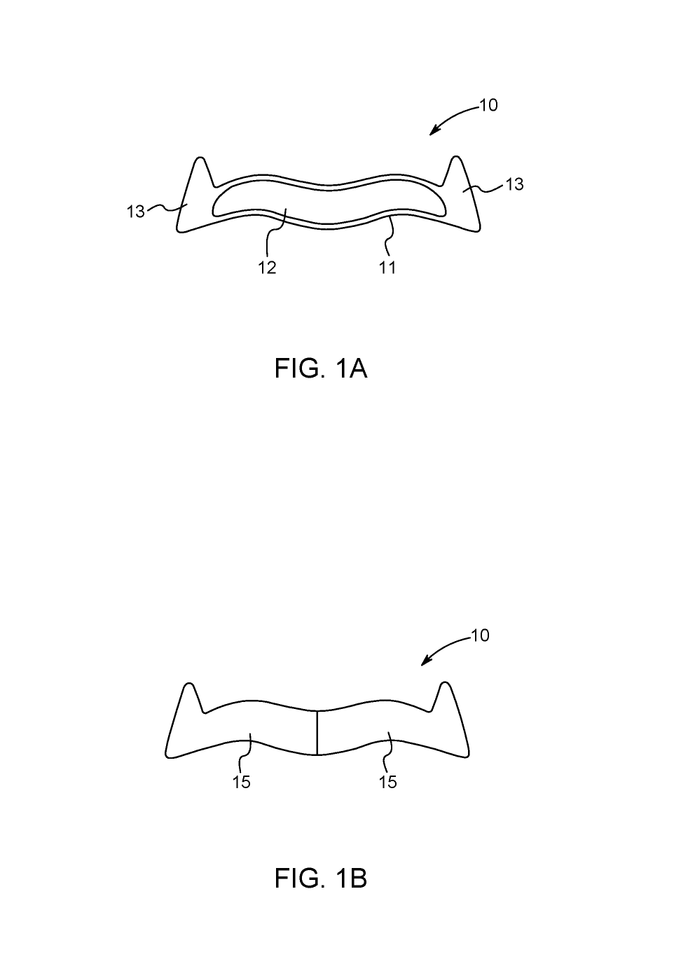

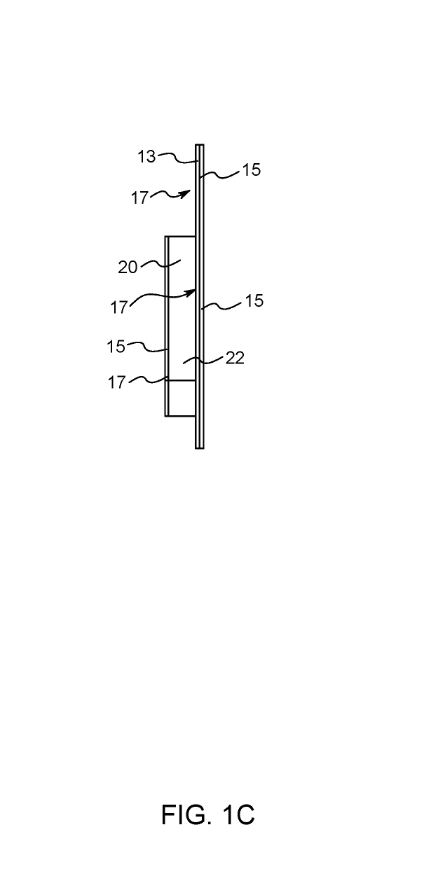

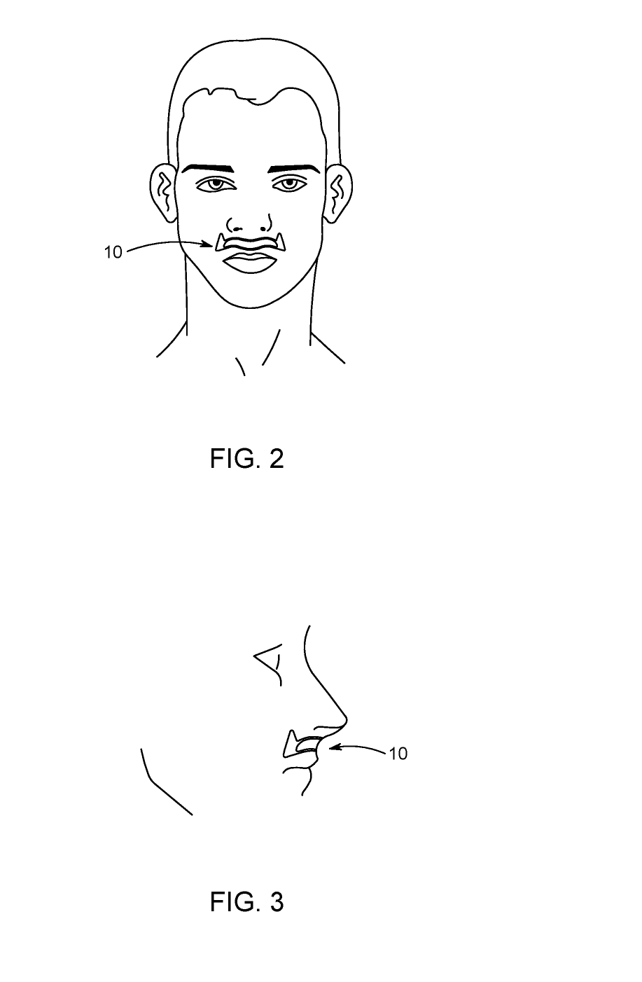

Referring now to the drawings and in particular FIGS. 1A, 1B, 1C, 2, and 3, an aromatherapy adhesive strip 10 is shown having a strip body 11 that includes a front side, FIG. 1A and a rear side, FIG. 1B. On the front side is an absorbent center 12 which is positioned between two ends 13. A rear side 15 is substantially coated with or otherwise includes an adhesive which allows it to removably attached to a user's skin. The rear side 15 includes a release paper 17 that covers an adhesive surface of the rear side 15. The rear side 15 contacts a user's skin during use. In one embodiment, the strip body 11 is constructed out of a flexible, non absorbent material such as plastic or a coated paper and the adhesive defines an acrylate.

In one embodiment, the absorbent center 12 defines a cotton pad 20, thereby allowing it to absorb essential oils and other liquid aromatics 22 and slowly diffuse the scent of the absorbed material over time. Because the absorbent center 12 is surrounded by the non-absorbent strip material, any absorbed material is able to be retained in place in the absorbent center. The absorbent center extends between the ends 13 and includes a center curve at the top. Further, the preferred embodiment includes 100% cotton material in the cotton pad 20.

In some embodiments, the absorbent center 12 is positioned in a depression in the surface of the strip body 11, thereby improving the ability of the absorbent center 12 to retain liquid aromatics 22 without them spilling onto strip body 11.

In addition, the strip body 11 lies between ends 13 and helps to direct the scents from an aromatic absorbed into the absorbent center 12 to the nose of the user, limiting the spread of such scents off the side of the strip body.

The instant invention has been shown and described herein in what is considered to be the most practical and preferred embodiment. It is recognized, however, that departures may be made therefrom within the scope of the invention and that obvious modifications will occur to a person skilled in the art.

What is claimed is:

1. An aromatic adhesive strip comprising:
a. a first end;
b. a second end;
c. an absorbent center extending between the first end and second end;
d. an adhesive surface on a rear side of the adhesive strip; and
e. a release paper covering the adhesive surface, where the release paper is adapted to be removed to apply the adhesive strip to an upper lip of a user.

2. The aromatic adhesive strip according to claim 1, where the absorbent center includes a cotton pad.

3. The aromatic adhesive strip according to claim 2, wherein the cotton pad includes a liquid aromatic oil.

4. The aromatic adhesive strip according to claim 3, where the liquid aromatic oil is adapted to diffuse a scent over a predetermined time period.

5. The aromatic adhesive strip according to claim 1 where the absorbent center is surrounded by non-absorbent strip material.

6. The aromatic adhesive strip according to claim 5, where the non-absorbent material is adapted to retain the absorbent center in place.
